(19) United States
(12) Patent Application Publication
(10) Pub. No.: US 2024/0161287 A1
(43) Pub. Date: May 16, 2024

(54) MEDICAL DEVICE INSPECTION SYSTEM WITH EXTERNAL INSPECTION DEVICE

(71) Applicant: Clarus Medical LLC, Minneapolis, MN (US)

(72) Inventors: Kristin Sundet PAVEK, Golden Valley, MN (US); Scott Allen SUNDET, Edina, MN (US); Andrew R. SUNDET, Excelsior, MN (US)

(73) Assignee: Clarus Medical LLC, Minneapolis, MN (US)

(21) Appl. No.: 18/494,573

(22) Filed: Oct. 25, 2023

Related U.S. Application Data
(60) Provisional application No. 63/380,766, filed on Oct. 25, 2022.

(51) Int. Cl. G06T 7/00; G01N 21/88; G01N 21/952; G01N 21/954; G06V 10/82; H04N 7/18; H04N 17/00; H04N 23/50

(52) U.S. Cl. CPC G06T 7/0012; G01N 21/8851; G01N 21/952; G01N 21/954; G06V 10/82; H04N 7/185; H04N 17/002; H04N 23/555; G06T 2207/20084; G06T 2207/30021; G06V 2201/034

(57) ABSTRACT

A medical device inspection system includes an external inspection device. In some embodiments the medical device inspection system includes an external inspection device and an internal inspection device. The medical device inspection system can be used to inspect a medical device for abnormalities.

### CROSS-REFERENCE TO RELATED APPLICATION(S)

This application claims priority to U.S. Application No. 63/380,766, filed on Oct. 25, 2022, entitled MEDICAL DEVICE INSPECTION SYSTEM WITH EXTERNAL INSPECTION DEVICE, the disclosure of which is hereby incorporated by reference in its entirety.

### BACKGROUND

Millions of medical devices are used in hospitals throughout the world every day. With the continuing advancement of medical and surgical procedures over time, one of the trends for many years is toward minimally invasive procedures performed through smaller incisions or even through the body's natural orifices. Examples of this trend include arthroscopic surgery, transcatheter aortic valve replacement (“TAVR”), natural orifice transluminal endoscopic surgery (“NOTES”), robotic surgery and many others. Many of these procedures involve the use of long, flexible catheter instruments, long, thin, rigid, instruments with lumens, and/or long, flexible endoscopes for visualizing the procedure. Additionally, endoscopes are used in countless different diagnostic and therapeutic procedures in many parts of the body.

One of the challenges with the use of endoscopes, fiber scopes, catheter-based medical/surgical instruments and other long, thin, reusable instruments is how to properly and effectively clean them, especially their inner lumens. Many endoscopes and other instruments are too expensive to be disposable and so must be reused. And long, small-diameter, flexible instruments can be extremely hard to clean on the inside. They are also hard to inspect on the inside. Not only can flexible instruments collect bacteria and other contaminants, but they can also crack or become otherwise permanently deformed during use, for example when the instrument is bent or kinked. These instruments are typically processed in a cleaning facility located within the hospital, by workers with very little training. To inspect the inside of such instruments, a small, flexible scope is inserted and advanced through the lumen(s) of the device, so that contaminants and damage can be seen. It can be difficult, however, for the person doing the inspection to effectively identify contaminants and internal damage to the device. Thus, the inspection process can be labor intensive and sometimes ineffective. It can also be hard to find a scope small enough to fit through the lumens of some medical devices while allowing for adequate visualization. Additionally, once contamination of an endoscope or catheter lumen (or similar inner portion of a medical device) is identified, it can often be difficult to adequately clean and/or decontaminate the lumen.

### SUMMARY

In general terms, this disclosure is directed to a medical device inspection system. In some embodiments, and by non-limiting example, the medical device inspection system includes an external inspection device. Some embodiments further include an internal inspection device.

One aspect is a medical device inspection system comprising: an internal inspection device configured to inspect an interior of a medical device; and an external inspection device configured to inspect an exterior of the medical device.

A further aspect is an external inspection system for inspecting an exterior of a medical device, the external inspection system comprising an external inspection device, the external inspection device comprising: a body having an interior surface defining an interior space, wherein the interior space is configured for the medical device to pass therethrough during an inspection of the medical device; and a camera supported by the body and directed toward the interior space to capture images of the exterior of the medical device during the inspection.

Yet another aspect is a method of inspecting a medical device, the method comprising: capturing images of an interior of a medical device; capturing images of an exterior of the medical device; and inspecting the medical device for abnormalities using the images of the interior of the medical device and the images of the exterior of the medical device.

### BRIEF DESCRIPTION OF THE DRAWINGS

FIG. 1 is a schematic block diagram illustrating an example medical device inspection system.

FIG. 2 is a block diagram illustrating an example of an external inspection device.

FIG. 3 shows an internal inspection device.

FIG. 4 is a schematic block diagram illustrating an example user interface of a computing device.

FIG. 5 illustrates an example computing device.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

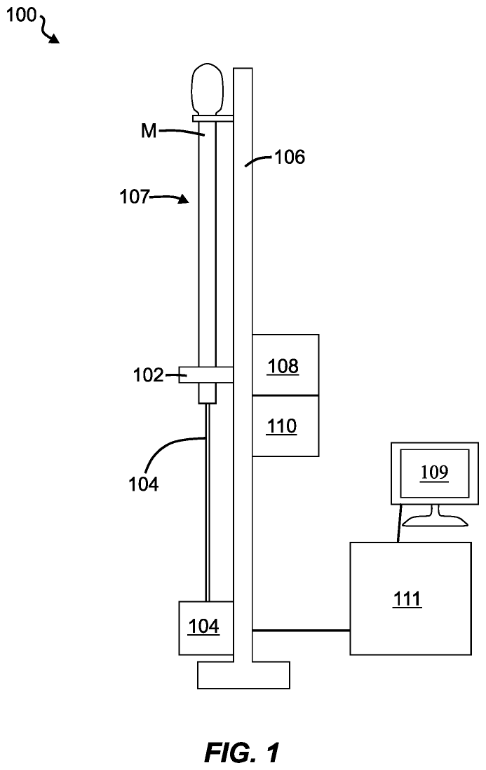

FIG. 1 is schematic block diagram illustrating an example medical device inspection system 100 including at least an external inspection device 102. In the illustrated example, the medical device inspection system 100 includes an external inspection device 102, an internal inspection system 104, and a support structure 106. The support structure includes a medical device holder 107. Some embodiments further include an advancement device 108, a position detector 110, a computing device 111, or combinations thereof. An example medical device M is also shown.

The present disclosure relates to inspection of a medical device M, which may be one of various different types of medical devices, several of which are discussed in the background. Some medical devices have an elongated flexible body and may include one or more internal orifices. Examples include endoscopes, fiber scopes, catheter-based medical/surgical instruments, and other reusable instruments.

The medical device inspection system 100 operates to inspect a medical device for abnormalities. Abnormalities can include, for example, debris, damage (e.g., gouges, kinks, cracks), discoloration, moisture (e.g., water droplets), contaminants (e.g., biofilms or biological material), and the like.

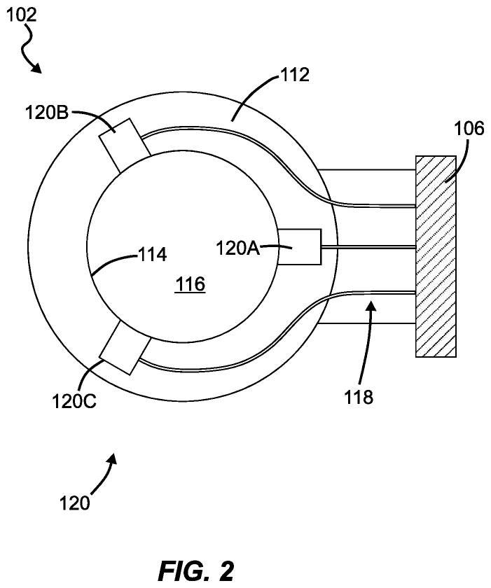

An external inspection device 102 is arranged and configured to inspect an exterior of the medical device M. An example of the external inspection device 102 is illustrated and described in further detail with reference to FIG. 2.

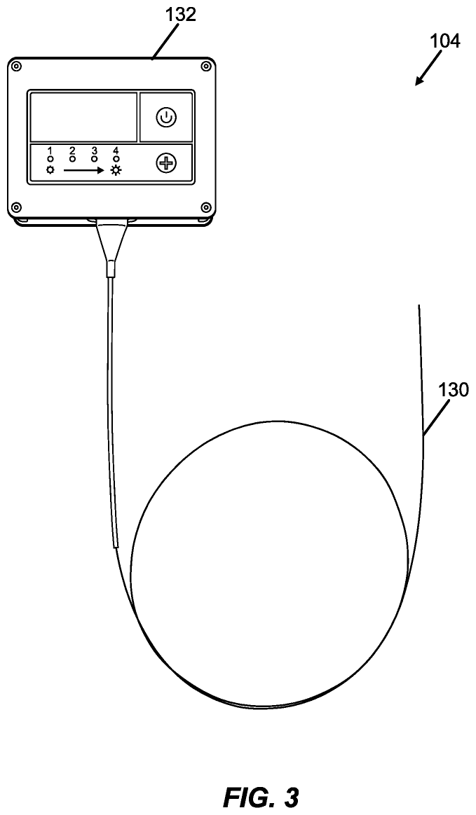

An internal inspection system 104 is arranged and configured to inspect an interior of the medical device M. An example of the internal inspection system 104 is illustrated and described in further detail with reference to FIG. 3.

A support structure 106 is provided in some embodiments to support components of the medical device inspection system 100, such as the external inspection device 102 and the internal inspection device 104. In this example the support structure 106 is shown in a configuration to support the medical device M in a vertical orientation. In another possible embodiment, the support structure 106 can be configured to support the medical device M in a horizontal position. For example, the support structure could be configured to be placed on a table, or could include its own table or other horizontal support structure. Other configurations are also possible.

In some embodiments the support structure 106 includes a medical device holder 107. The medical device holder 107 can include one or more clamps, clips, hangers, brackets, arms, or other structures suitable to support the medical device in a desired position. The medical device holder 107 permits the medical device to be easily inserted and removed from the medical device holder 107 by an operator.

Some embodiments include an advancement device 108. In some embodiments, the advancement device 108 is motorized using one or more motors. In another possible embodiment, the advancement device 108 uses friction to slow movement of the medical device M under the force of gravity. In this way, some embodiments of the advancement device 108 can provide movement without requiring motors. The advancement device 108 operates to move one or both of the medical device M and the internal inspection device 104 such that the internal inspection device moves relative to the medical device. Movement can be in either direction, or both directions. Similarly, the advancement device 108 operates, in some embodiments, to move one or both of the medical device M and the external inspection device 102, such that the external inspection device moves relative to the medical device M. In some embodiments the advancement device 108 moves the medical device M while the external inspection device 102 and the internal inspection device 104 remain stationary.

In some embodiments the support structure 106 includes one or more tracks. The advancement device is coupled one or more of: the medical device holder 107, the external inspection device 102, or the internal inspection device 104, to cause such components to move along the one or more tracks.

Some embodiments do not include an advancement device 108. For example, an operator can manually move the external inspection device 102 relative to the medical device M, and/or the internal inspection device 104 relative to the medical device M.

With respect to the internal inspection device 104, in some embodiments the internal inspection device 104 is inserted and advanced in a forward direction through the medical device M during the inspection process. In another embodiment, the internal inspection device 104 is first inserted or advanced through the medical device M, and is subsequently withdrawn from the medical device M while the inspection occurs. In yet another possible embodiment, inspection can take place during both insertion and withdrawal of the internal inspection device 104.

A position detector 110 is provided in some embodiments to detect relative positioning between the medical device M, external inspection device 102, and/or the internal inspection device 104. The position detector 110 can be used, for example, to determine a position of the internal inspection device 104 as a depth within the medical device M as measured from an opening, or to determine how far the external inspection device is from the same opening. Some embodiments do not include a position detector 110. For example, in some embodiments position measurements can be taken manually by an operator. In some embodiments the position measurements are entered by the operator into the computing device 111. Further, in some embodiments the position detector 110 can be part of the advancement device 108. For example, a stepper motor can be configured to precisely control movements, and as a result, positions can be calculated based on operation of the advancement device.

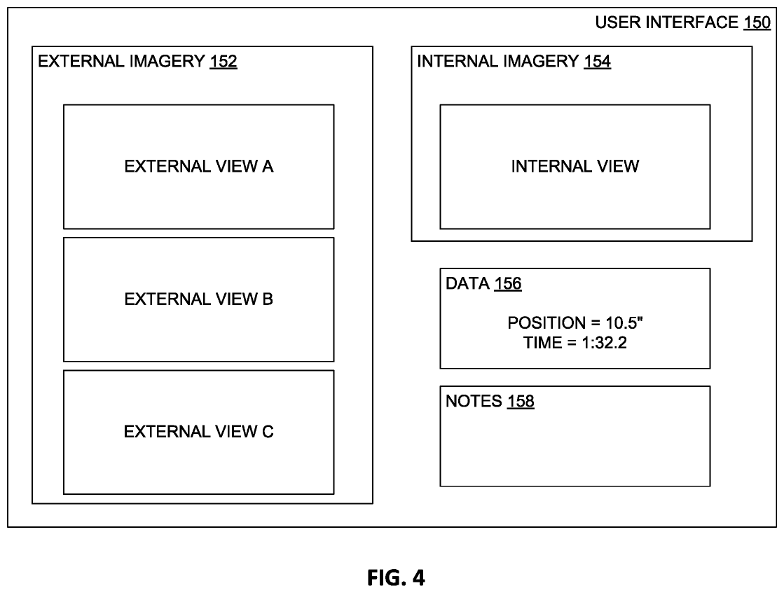

Some embodiments include one or more computing devices 111. The computing device 111 can operate to control one or more operations of the medical device inspection system 100, and/or to collect, analyze, and/or store data from the other components of the medical device inspection system 100. For example, images and other data from the external inspection device 102, internal inspection device 104, advancement device, or position detector can be provided to the computing device 111. In some embodiments the computing device 111 controls and coordinates the operation of the internal inspection device 104 and the external inspection device 102. An example user interface display provided by the computing device 111 is illustrated and described in further detail with reference to FIG. 4.

The medical device inspection system 100 can include one or more inspection devices. Some embodiments include an external inspection device 102. Other embodiments include an internal inspection device 104. Some embodiments include both. Further embodiments may have additional inspection devices.

The inspection devices can be operated one at a time, one after the other, or simultaneously, in any order. In one example, a camera of the internal inspection device 104 can be positioned at one end of the medical device M, while the external inspection device 102 is simultaneously positioned at the same end. The inspection devices 102 and 104 can then be moved at the same speed along the length of the medical device M. In this way the inspection devices 102 and 104 simultaneously inspect the inside and outside at the same position relative to the length of the medical device. In an alternative embodiment, the inspection devices can be spaced from one another, such that they scan separate portions of the medical device at any given time. In yet another alternative embodiment, the exterior and internal inspection devices 102 and 104 can be operated one at a time, to scan the interior and the exterior separately.

FIG. 2 is a block diagram illustrating an example of an external inspection device 102. In this example, the external inspection device 102 includes a body 112 with an interior surface 114, which defines an interior space 116. One or more cameras 120 (including 120A, 120B, and 120C) are supported by the body and are directed inward toward the interior space 116.

The body 112 defines an interior space through which the medical device M can pass. The medical device would preferably be arranged so that it is approximately centered within the interior space 116 (e.g., concentric, though non-circular shapes are also possible). In this example, the body 112 is supported by the support structure 106. As discussed herein, the body 112 may also be movable along the support structure by the advancement device.

In some embodiments, the cameras are supported at fixed (though possibly adjustable) positions about the interior surface 114 of the body, and have a field of view arranged inward toward the interior space 116. One or more cameras can be included. In certain examples, at least two cameras are needed—one on each side—to image all exterior sides of the medical device M. Although it is also possible to use one or more mirrors or other optical features to reduce the number of required cameras. Some embodiments include three or more cameras. The illustrated example shows three cameras 102A, 102B, and 102C. Camera 102A captures imagery of an external view A, camera 102B captures imagery of an external view B, and camera 102C captures imagery of an external view C.

In some embodiments, a single camera is supported at an interior surface 114 of the body to image all exterior sides of the medical device M. In one example, the external inspection device is configured to rotate the camera about the exterior of the medical device. For example, the body 112 is rotatably mounted to the support structure 106 such that the camera captures the exterior sides of the medical device when the body 112 completes one rotation with the camera having a field of view arranged inward toward the interior space 116.

Cameras can also be arranged at various possible angles, as desired, such as in the direction of the length of the medical device M. Such an arrangement may more closely match the corresponding imagery captured by the internal inspection device 104. Such angles result in a perspective view that may provide better views for detecting certain abnormalities that may be more difficult to detect from a pure side view.

In some embodiments the external inspection device 102 includes one or more light sources. Light sources can be arranged similar to the cameras in the body 112, or can be offset along a length of the medical device and arranged to illuminate the interior space. One or more light sources can be provided, and light sources can be of multiple different types in some embodiments. For example, the light sources can include a visible light source and an ultraviolet (UV, such as UV-C) light source in some embodiments. The light sources can be independently operable.

Some embodiments include wiring 118 to route power and/or data signals. In one example, the wiring 118 extends through the body and is electrically connected to one or more electronic components therein, such as the one or more cameras 120. In some embodiments the wiring 118 is then routed through the body and out into the support structure 106. The wiring can ultimately convey power from a power source, and/or digital signals (such as from the one or more cameras 120) to the computing device 111.

Although the external inspection device 102 is illustrated as containing wiring 118 connecting it to the support structure, such wiring 118 is not required by all embodiments. For example, the external inspection device 102 and/or the support structure 106 can include their own power sources, such as batteries. Similarly, data signals may be transferred wirelessly using a wireless transceiver, such as using Wi-Fi, Bluetooth, a cellular network, satellite communication, and/or other radio-frequency signals or technology.

FIG. 3 shows an example internal inspection device 104. In this example, the internal inspection device 104 includes an inspection scope 130 including a control unit 132. An example of an inspection scope 130 is a borescope, such as a fiber scope. The inspection scope typically includes a light source and a camera. The internal inspection device 104 has a long narrow fiber that can be inserted and removed through the center of the medical device, and operates to capture images of the interior of the medical device M. Examples of inspection scopes are disclosed in various patent applications by Clarus Medical, LLC including US 2019/0224357, filed on Jan. 22, 2019; US 2019/0282327, filed on Feb. 19, 2019; US 2022/0080469, filed on Sep. 10, 2021; and US 2022/0240767, filed on Feb. 3, 2022, the disclosures of which are hereby incorporated by reference in their entireties.

The inspection scope 130 is configured to inspect the interior of the medical device M and generate inspection data. The inspection data includes any one or more of image data, video data, inspection metadata, operational data for documenting the operation of the inspection system, or any combination thereof.

In one example, the inspection data includes the images (including optionally video) taken by the inspection scope 130. Another example of the inspection data is time stamps identifying a date and/or time at which the images were taken. Another example of the inspection data is position data from the position tracker 138 identifying the positions at which the images were taken. The data can be stored in a memory device. In some embodiments, the data is stored at one or multiple databases which may consist of or include one or multiple third-party databases simultaneously.

Inspection data can also include operational data. Operational data is data documenting the operation of the inspection system during the inspection. A variety of optional data can be collected. For example, position and time data can be collected. Position and time data may be associated with captured images or a video clip or recording. Speed data can be collected identifying relative speed of movement of the inspection scope 130 relative to the medical device. The images can be evaluated to determine a quality of the images, and data documenting the quality of the images can be stored.

The operational data may also include information about the one or more operators (e.g., technicians) involved in one or more steps of the inspection process, such as date and time of when the step occurred, and an identification of the operator, such as a name or identification number. Similarly, in some examples, information about a cleaning or inspection station (e.g., workstation) or a physical location within a building can be collected and stored. In these examples, such information can be manually entered by the operators, or determined by various scanning (e.g., barcode, RFID, etc.) or position determining processes.

In some embodiments the operational data includes information about the inspection system and/or inspection scope 130 that is being used to perform the inspection and scan. The information can include a manufacturer's name, model number, and/or an identification number. The information can also include inspection system characteristics (e.g., length, diameter, etc.) and capabilities (e.g., cleaning features (e.g., a brush), disinfecting features (e.g., UV light)), and whether such capabilities were utilized. If so, which ones, when, at what position(s), how much, and/or for how long. For example, data can be collected regarding specifically what wavelength(s) of UV light were used (e.g., UV-C), what intensity, where it was used, and a length of time of exposure, or other dosage measurement.

The storage of inspection data can be temporary or permanent. For example, in some embodiments, inspection data is stored for processing and unneeded data can be subsequently deleted. In other embodiments, inspection data is stored regardless of other operations.

Inspection data can be stored in a variety of manners, such as in one or more files, in a database, and the like. In some embodiments images are associated with corresponding data, such as position and time data. The data can be stored in a database and associated with the images in the database. In another possible embodiment the data can be stored in metadata of the image (e.g., time and location fields of the image) as inspection metadata, or in the file name. For example, the file name can be a combination of one or more of a medical device identifier, date, time, position, and/or other data. In some embodiments, the inspection data can be saved at another system and/or database.

In the same way that the inspection data and operational data can be collected with respect to the inspection scope 130, the same or similar data can be collected with respect to the external inspection device 102 described herein.

The inspection scope 130 can be supported by the support structure 106. For example, the support structure 106 can include one or more clamps, clips, hangers, brackets, arms, or other structures suitable to support and guide the inspection scope 130 as it moves into or out from the interior of the medical device M.

In some examples, the computing device 111 (shown in FIG. 1) includes an inspection analyzer for analyzing the inspection data to identify possible abnormalities of the medical device. In some embodiments the inspection analyzer is or includes one or more software applications. In some embodiments the inspection analyzer is or includes a neural network, such as a convolutional neural network (CNN), which may operate on one or more computing devices, and may involve one or remote computing devices. An example of the neural network is a deep neural network.

In some embodiments the inspection analyzer includes an abnormality detector. The abnormality detector can be or include one or more machine learning algorithms (e.g., artificial intelligence) including one or more machine learning models trained to detect or predict whether an abnormality is present. In some embodiments the abnormality detector performs image analysis. In some embodiments the abnormality detector performs object recognition. In some embodiments the abnormality detector is or includes an image classifier. The abnormality detector can be or include one or more machine learning algorithms that are supervised or unsupervised machine learning algorithms. In certain examples, the abnormality detector automatically detects abnormalities in the medical device M by processing the inspection data.

In some embodiments the abnormality detector is trained on a set of training data. The training data can include positive training examples, negative training examples, or positive and negative training examples, and can include interior images and/or exterior images of the medical device M. The examples can include images of medical devices without abnormalities, and images of medical devices with abnormalities. The training examples can be labeled with certain data, such as whether an abnormality is present or not, and/or a type or class of abnormality. A variety of abnormalities are possible, including for example debris, damage, discoloration, droplets (moisture), etc.

In some embodiments, medical device inspection system 100 includes a position tracker for determining a relative position of at least one of: (i) the external inspection device 102 with respect to the medical device, (ii) the internal inspection device 104 (e.g., inspection scope) with respect to the medical device, or both. The position tracker generates quantitative data, qualitative data, or both, in various possible embodiments. The inspection data includes data generated from the position tracker. In some embodiments, a quantitative position can be a measurement. An example of a measurement is a distance from an opening in (or from another reference point of) the medical device. For example, the position tracker can use an opening in the medical device as an origin location, and then measure movement of the tip of the inspection scope 130 into the medical device, relative to the origin location (e.g., 1 cm, 2 cm, 3 cm, 4 cm, . . . etc.). Examples of qualitative positions can be defined with respect to particular parts or locations within the medical device M, such as at or near to a particular hotspot, or at or near to a particular part, edge, or other location. Such qualitative positions can also be identified using quantitative measurements, or can be identified using other techniques, such as image recognition. The position tracker can use both quantitative and qualitative positions in some embodiments.

In some embodiments the position tracker operates to identify a position when an image is taken, so that the precise location of the medical device where the image was taken is known. The position tracker can also be used to measure a speed of the relative movement between the inspection scope 130 and the medical device. Speed can also be computed based on detected positions and a duration of time that elapsed between those positions.

The control unit 132 may be a specific component of the internal inspection device 104, as shown, or alternatively can be the computing device 111, such that other components (such as the external inspection device 102, advancement device 108, and/or the position detector 110) also connect to it.

FIG. 4 is a schematic block diagram illustrating an example user interface 150 of a computing device, and also illustrates example imagery captured by the medical device inspection system 100. In this example, the user interface 150 includes external imagery display 152 and internal imagery display 154. Some embodiments further include data display 156, and user input regions 158.

The external imagery display 152 displays the one or more external images captured by the one or more cameras 120. In this example, the display 152 includes three separate displays (View A, View B, and View C), one for each of cameras 102A, 102B, and 102C.

In some embodiments image processing is performed to merge multiple images into a single (panoramic style) image and corresponding view.

The internal imagery display 154 displays the one or more internal images captured by the one or more cameras of the internal inspection device 104.

Data can also be displayed, such as the position at which the images were captured, and a time when the images were captured.

In some embodiments one or more user input regions 158 are provided, such as to document details of the inspection or status of the medical device.

In some embodiments, the user interface 150 includes one or more reference images. For example, the user interface 150 can display a reference image for an interior of the medical device M. As another example, the user interface 150 can display a reference image for an exterior of the medical device M. In some embodiments, the user interface displays both. The reference images can show, for example, what the medical device M should look like absent any abnormalities, or in another example, what the medical device M looked like during a previous inspection. The reference images can be used by an operator or by the inspection analyzer to compare the inspection images obtained during the inspection with the reference images.

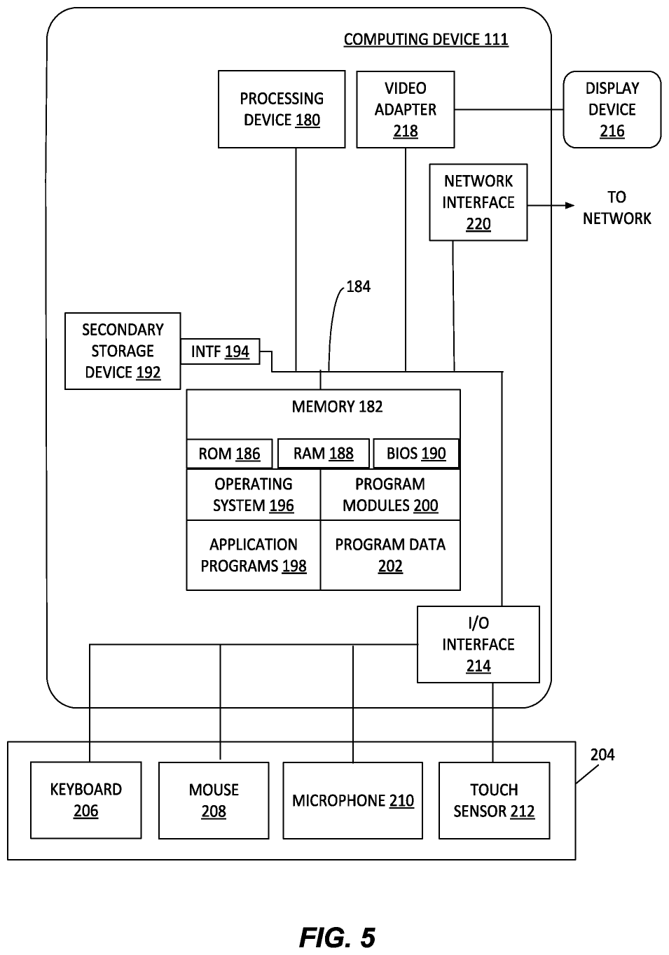

FIG. 5 illustrates an exemplary architecture of a computing device that can be used to implement aspects of the present disclosure, including any of the plurality of computing devices disclosed herein. The computing device may be local to or remote from the inspection scope, and to one or more other computing devices. The computing device may be a personal computer or a server computing device. The computing device illustrated in FIG. 5 can be used to execute the operating system, application programs, and software modules (including the software engines) described herein.

The computing device 111 includes, in some embodiments, at least one processing device 180, such as a central processing unit (CPU). A variety of processing devices are available from a variety of manufacturers, for example, Intel or Advanced Micro Devices. In this example, the computing device 111 also includes a system memory 182, and a system bus 184 that couples various system components including the system memory 182 to the processing device 180. The system bus 184 is one of any number of types of bus structures including a memory bus, or memory controller; a peripheral bus; and a local bus using any of a variety of bus architectures.

Examples of computing devices suitable for the computing device 111 include a server computer, a desktop computer, a laptop computer, a tablet computer, a mobile computing device (such as a smart phone, an iPod® or iPad® mobile digital device, or other mobile devices), or other devices configured to process digital instructions.

The system memory 182 includes read only memory 186 and random-access memory 188. A basic input/output system 190 containing the basic routines that act to transfer information within computing device 111, such as during start up, is typically stored in the read only memory 186.

The computing device 111 also includes a secondary storage device 192 in some embodiments, such as a hard disk drive, for storing digital data. The secondary storage device 192 is connected to the system bus 184 by a secondary storage interface 194. The secondary storage devices 192 and their associated computer readable media provide nonvolatile storage of computer readable instructions (including application programs and program modules), data structures, and other data for the computing device 111.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media. Additionally, such computer readable storage media can include local storage or cloud-based storage.

A number of program modules can be stored in secondary storage device 192 or memory 182, including an operating system 196, one or more application programs 198, other program modules 200 (such as the software engines described herein), and program data 202. The computing device 111 can utilize any suitable operating system, such as Microsoft Windows™, Google Chrome™, Apple OS, and any other operating system suitable for a computing device.

In some embodiments, a user provides inputs to the computing device 111 through one or more input devices 204. Examples of input devices 204 include a keyboard 206, mouse 208, microphone 210, and touch sensor 212 (such as a touchpad or touch sensitive display). Other embodiments include other input devices 204. The input devices are often connected to the processing device 180 through an input/output interface 214 that is coupled to the system bus 184. These input devices 204 can be connected by any number of input/output interfaces, such as a parallel port, serial port, game port, or a universal serial bus. Wireless communication between input devices and the interface 214 is possible as well, and includes infrared, BLUETOOTH® wireless technology, 802.11a/b/g/n, cellular, or other radio frequency communication systems in some possible embodiments.

In this example embodiment, a display device 216, such as a monitor, liquid crystal display device, projector, or touch sensitive display device, is also connected to the system bus 184 via an interface, such as a video adapter 218. In addition to the display device 216, the computing device 111 can include various other peripheral devices (not shown), such as speakers or a printer.

When used in a local area networking environment or a wide area networking environment (such as the Internet), the computing device 111 is typically connected to the network through a network interface 220, such as an Ethernet interface. Other possible embodiments use other communication devices. For example, some embodiments of the computing device 111 include a modem for communicating across the network.

The computing device 111 typically includes at least some form of computer readable media. Computer readable media includes any available media that can be accessed by the computing device 111. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 111. Computer readable storage media does not include computer readable communication media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 5 is also an example of programmable electronics, which may include one or more such computing devices, and when multiple computing devices are included, such computing devices can be coupled together with a suitable data communication network so as to collectively perform the various functions, methods, or operations disclosed herein.

The various embodiments described above are provided by way of illustration only and should not be construed to limit the claims attached hereto. Those skilled in the art will readily recognize various modifications and changes that may be made without following the example embodiments and applications illustrated and described herein, and without departing from the full scope of the following claims.

What is claimed is:

1. A medical device inspection system comprising:
an internal inspection device configured to inspect an interior of a medical device; and
an external inspection device configured to inspect an exterior of the medical device.

2. The medical device inspection system of claim 1, wherein the internal inspection device comprises an internal inspection scope including at least one camera, the internal inspection scope configured to capture images of the interior of the medical device.

3. The medical device inspection system of claim 1, wherein the external inspection device comprises at least one camera configured to capture at least one image of the exterior of the medical device.

4. The medical device inspection system of claim 3, wherein the external inspection device is configured to rotate the at least one camera about the exterior of the medical device.

5. The medical device inspection system of claim 4, wherein the external inspection device comprises a body rotatably mounted to a support structure for rotating the at least one camera about the exterior of the medical device.

6. The medical device inspection system of claim 1, further comprising a support structure configured to support the internal inspection device and the external inspection device during the inspection of the interior and the exterior of the medical device.

7. The medical device inspection system of claim 1, further comprising a computing device that controls and coordinates the operation of the internal inspection device and the external inspection device.

8. The medical device inspection system of claim 1, wherein the internal inspection device comprises a borescope.

9. An external inspection system for inspecting an exterior of a medical device, the external inspection system comprising an external inspection device, the external inspection device comprising:
a body having an interior surface defining an interior space, wherein the interior space is configured for the medical device to pass therethrough during an inspection of the medical device; and
a camera supported by the body and directed toward the interior space to capture images of the exterior of the medical device during the inspection.

10. The external inspection system of claim 9, further comprising a position tracker for determining a relative position of the external inspection device with respect to the medical device.

11. The external inspection system of claim 9, further comprising a computing device comprising an inspection analyzer, wherein the inspection analyzer analyzes the images of the exterior of the medical device to identify possible abnormalities of the medical device.

12. The external inspection system of claim 11, wherein the inspection analyzer further comprises an abnormality detector that automatically identifies possible abnormalities of the medical device.

13. The external inspection system of claim 12, wherein the abnormality detector automatically detects the possible abnormalities of the medical device by processing the images of the exterior of the medical device.

14. The external inspection system of claim 11, wherein the inspection analyzer comprises one or more trained machine learning models operating on one or more neural networks.

15. The external inspection system of claim 8, further comprising a wireless transceiver for wirelessly transmitting data from the camera.

16. The external inspection system of claim 15, further comprising at least one battery power source for powering the camera and the wireless transceiver.

17. A method of inspecting a medical device, the method comprising:
capturing images of an interior of a medical device;
capturing images of an exterior of the medical device; and
inspecting the medical device for abnormalities using the images of the interior of the medical device and the images of the exterior of the medical device.

18. The method of claim 17, further comprising:
generating inspection data including the images of the interior of the medical device and the images of the exterior of the medical device;
analyzing the inspection data using a machine learning model;
generating analysis data based on the analysis of the inspection data; and
generating one or more outputs based on the analysis data.

19. The method of claim 18, wherein the inspection data includes any one or more of:
(a) image data;
(b) video data;
(c) inspection metadata;
(d) operational data documenting the operation of the inspection system; or
(e) any combination of (a), (b), (c) and (d).

20. The method of claim 18, wherein the analysis data includes a prediction of whether the medical device may have an abnormality.
